SEQUENCE LISTING (3 sequences)

SEQ ID NO: 1 - protein, 119 aa, Artificial Sequence
  NDMC-ASK1, which is an  anti-Gd-IgA1 monoclonal antibody selecting from  hybridoma clones via the laboratory
EVQLQQSGPELVKPGASMKISCKASGYSFTGYTMNWVKQSRGKNLEWIGLINPYNGVTSYNQKFKGKATLTVDKSSITAYMEFLSLTSEDSAVYYCARSGYGNYALAYWGQGTLVTVSA

SEQ ID NO: 2 - protein, 117 aa, Artificial Sequence
  NDMC-ASK2, which is an  anti-Gd-IgA1 monoclonal antibody selecting from  hybridoma clones via the laboratory
QIQLVQSGPELKKPGETVKISCKASGYTFTNCGMNWVRQAPGKGLKWMGWINTYTGKPTYADDFKGRFAFSLETSASTAYLQINNLKNEDMATYFCTKYGYDPFDYWGQGTTLTVSS

SEQ ID NO: 3 - protein, 119 aa, Artificial Sequence
  NDMC-ASK3, which is an  anti-Gd-IgA1 monoclonal antibody selecting from  hybridoma clones via the laboratory
QVQLQQSGPELVKPGASVKISCKASGYTFTSYYIHWVKQRPGQGLEWIGYIYPRDGSTNYNEKFKGKATLTADTSSSTAYMQLSSLTSEDSAVYFCARQLGLPAWFAYWGQGTLVTVSA

(19) United States
(12) Patent Application Publication
(10) Pub. No.: US 2022/0275106 A1
(43) Pub. Date: Sep. 1, 2022

(54) MOUSE MONOCLONAL ANTIBODIES AGAINST GALACTOSE-DEFICIENT IGA1,PREPARATION METHOD THEREOF, AND USE THEREOF

(71) Applicant: National Defense Medical Center, Taipei City (TW)

(72) Inventors: Ann CHEN, Taipei City (TW); Shuk-Man KA, Taipei City (TW)

(21) Appl. No.: 17/684,193

(22) Filed: Mar. 1, 2022

Related U.S. Application Data
(60) Provisional application No. 63/154,919, filed on Mar. 1, 2021.

(51) Int. Cl. C07K 16/42; G01N 33/68

(52) U.S. Cl. CPC C07K 16/4241; G01N 33/686; G01N 2800/347; G01N 2800/52

(57) ABSTRACT

The current invention provides high specificity mouse monoclonal antibodies, which can specifically bind to Gd-IgA as a novel non-invasive method for rapid diagnosing of IgAN subjects, can be applied to unravel the mechanisms of IgA nephropathy and establish therapeutical strategies.

### CROSS-REFERENCE TO RELATED APPLICATIONS

This application claims priority in U.S. Provisional Patent Application No. 63/154,919, filed Mar. 1, 2021, which is incorporated by reference in its entirety herein.

### FIELD OF THE INVENTION

The invention relates to a kit and method for detecting galactose-deficient IgA1 (Gd-IgA1), a kit and method for diagnosing IgA nephropathy (IgAN) in a subject.

### BACKGROUND OF THE INVENTION

IgA nephropathy (IgAN) is the most common cause of glomerulonephritis worldwide. Until now, an overall incidence of IgAN is about at least 2.5 per 100,000 individuals. Of such IgA nephropathy cases, 15 to 30% having a poor prognosis progress into renal failure.

Previous studies have shown that IgAN is closely related to the abnormality of O-glycosylation of galactose-deficiency in immunoglobulin A (Gd-IgA1). Abnormally glycosylated IgA forms immune complexes and deposits in the kidney glomerulus of the kidney, resulting in activation of immune cells to produce cytokines, and renal cell proliferation, leading to kidney failure. Unfortunately, there is no disease-specific treatment available for IgAN, and once uremia occurs, the patients will be only on dialysis treatment or receive kidney transplantation.

At present, the diagnosis of IgAN requires clinical characterization and invasive surgery, and pathological interpretation of renal tissue sections is performed to confirm the lesion. That is to say the diagnosis of IgA nephropathy requires a renal biopsy in combination with light microscopy, immunofluorescence and electron microscopy. Renal biopsy involves an invasive procedure and might cause complications such as internal hemorrhage.

Therefore, it is exceptionally urgent to develop an IgA1 glycan-based analysis using reagents with high readability and specificity.

### SUMMARY OF THE INVENTION

In view of the above-mentioned problem, the present invention provides a non-invasive diagnostic kit or/ and a non-invasive diagnostic method for rapid diagnosing of IgAN patients.

Another aspect of the present invention provides a strategy to apply a liquid biopsy for monitoring a biomarker in the blood from IgAN subjects using novel antibodies via immunoassay.

In one embodiment, the present invention also provides a kit for unraveling the mechanisms of IgA nephropathy and establishing therapeutical strategies.

In an alternative embodiment, the instant invention also provides a method for detecting galactose-deficient IgA1 in a subject, comprising: (a) obtaining a biological sample from a subject; and (b) using an antibody in an immunoassay to detect galactose-deficient IgA1 in the subject.

In certain embodiment, the present invention further provides a method of diagnosing IgA nephropathy in a subject, comprising, comprising: (a) using an antibody in an immunoassay; and (b) determining whether the antibody binds to a galactose-deficient IgA1, binding of the antibody to the galactose-deficient IgA1 indicating the subject has or is at risk of developing IgA nephropathy.

Accordingly in an aspect of the invention there is provided a method for monitoring a subject undergoing a treatment or therapy for an immune response to determine whether the subject is responsive to the treatment or therapy comprising detecting a level of expression, activity and/or function of a biomarker or a biomarker in the absence of the treatment or therapy and comparing the level of expression, activity and/or function of a biomarker in the presence of the treatment or therapy, wherein a difference in the level of expression, activity and/or function of the biomarker in the presence of the treatment or therapy indicates whether the patient is responsive to the treatment or therapy, wherein the biomaker is an galactose-deficient glycoform on hinge region of IgA1 and wherein the treatment or therapy is specific for an antibody

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The foregoing summary, as well as the following detailed description of the invention, will be better understood when reading in conjunction with the appended drawings.

FIG. 1A-1D is a schematic diagram of Binding test of monoclonal IgG antibody gainst Gd-IgA1 in Dot blot (FIG. 1A-1B) or Western blotting (FIG. 1C-1D). A Dot blotting or Western blotting was conducted via loading I-IgA1 and prepared enzymatic Gd-IgA1 after amplifying the numbers of Gd-IgA1-specific IgG monoclonals. Then the mouse monoclonal IgG antibody as the primary antibody was used in the present invention. The results showed the monoclonal IgG can significantly bind to enzymatically prepared Gd-IgA1 compared to I-IgA1 when loading with the same amount of IgA1.

FIG. 2A-2B show serum levels of Gd-IgA1 by NDMC-ASK2-based ELISA. As illustrated by NDMC-ASK2, one monoclonal mouse antibody was created to show that Serum Gd-IgA1 levels in patients with IgAN, other renal diseases, include DN, LN, and non-renal disease—SLE, were determined by NDMC-ASK2-based ELISA. It can well discriminate IgAN patients from healthy controls (HCs) with treating sera with neuraminidase for desialylation of glycans (FIG. 2A). ROC analysis of serum samples was treated with neuraminidase, the area under the ROC curve (AUC-ROC) of Gd-IgA1 levels to predict IgAN patient was 0.84 (FIG. 2B). HC, Healthy control; IgAN, IgA nephropathy; SLE, Systemic Lupus Erythematosus; DN, diabetic nephropathy; LN, lupus nephritis. AUC, area under the curve.

FIG. 2C show serum levels of Gd-IgA1 by NDMC-ASK2-based ELISA. As illustrated by NDMC-ASK2, one monoclonal mouse antibody was created to show that Serum Gd-IgA1 levels in patients with IgAN, and non-renal diseases, were determined by NDMC-ASK2-based ELISA. It can well discriminate IgAN patients from healthy controls (HCs) without or with treating sera with neuraminidase for desialylation of glycans (FIG. 2C). ****p<0.0001. HC, Healthy control; IgAN, IgA nephropathy.

FIG. 3A-3H depict that the analysis of serum Gd-IgA1 levels measured by ELISA. Compared between serum Gd-IgA1 levels were measured by NDMC-ASK2-anti-Gd-IgA1 ELISA and KM-55 ELISA using ROC curve analyses. Serum Gd-IgA1 levels in patients with IgAN and non-renal diseases were determined by KM55-Gd-IgA1 ELISA (FIG. 3A-3B). A ROC curve was drawn for serum Gd-IgA1 levels by KM55-Gd-IgA1 ELISA (FIG. 3C-3D). The same samples were measured for serum Gd-IgA1 levels by NDMC-ASK2-Gd-IgA1 ELISA (FIG. 3E-3F). A ROC curve was drawn for serum Gd-IgA1 levels by NDMC-ASK2-Gd-IgA1 ELISA (FIG. 3G-3H).

FIG. 4A-4B illustrates serum levels of Gd-IgA1-IgG autoantibody ICs by NDMC-ASK2-based ELISA. An assay for NDMC-ASK2 coating with 96 well plates and detected serum by anti-human IgG was established. A standard curve was made to define the amount of exposure on Gd-IgA1-IgG IC, using a human Gd-IgA1 protein. One unit is equal to 1μg Gd-IgA1 IgG IC. ****p<0.0001; Gd-IgA1, Galactose deficient IgA1; IgAN, IgA nephropathy; HC, health control; IC, immune complex.

FIG. 5A-5B illustrates that Combined NDMC-ASK2 and IC analysis increased sensitivity and specificity by ROC curve and logistic regression analyses. A combined NDMC-ASK2 and partial immune-complex with NDMC-ASK2 assay were established by combining ROC curve and logistic regression analyses, which helps increase the sensitivity to 0.906 although the specificity is 0.974.

FIG. 6A-6B illustrate that Serum Gd-IgA1 levels were measured by NDMC-ASK1-anti-Gd-IgA1 ELISA and ROC curve analyses. Serum Gd-IgA1 levels in patients with IgAN, and non-renal diseases were determined by NDMC-ASK1-Gd-IgA1 ELISA (FIG. 6A). A ROC curve was drawn for serum Gd-IgA1 levels by KM55-Gd-IgA1 ELISA (FIG. 6B). HC, Healthy control; IgAN, IgA nephropathy; SLE, Systemic Lupus Erythematosus; DN, diabetic nephropathy; LN, lupus nephritis. AUC, area under the curve.

FIG. 7A-7B illustrate that Serum Gd-IgA1 levels were measured by NDMC-ASK3-anti-Gd-IgA1 ELISA and ROC curve analyses. Serum Gd-IgA1 levels in patients with IgAN, and non-renal diseases were determined by NDMC-ASK3-Gd-IgA1 ELISA (FIG. 7A). A ROC curve was drawn for serum Gd-IgA1 levels by KM55-Gd-IgA1 ELISA (FIG. 7B). HC, Healthy control; IgAN, IgA nephropathy; SLE, Systemic Lupus Erythematosus; DN, diabetic nephropathy; LN, lupus nephritis. AUC, area under the curve.

FIG. 8A-8C depict that Mouse monoclonal antibodies (moAbs), binding affinity with glycopeptide epitopes. IgG mAb (including NDMC-ASK2 (FIG. 8A) and NDMC-ASK1 (FIG. 8B)) showed binding to glycopeptides, including T225, T228, S230, S232, T233, and T236, to which Tn antigen is attached in the IgA1 hinge region. IgM moAb (including NDMC-ASK3 (FIG. 8C)) showed binding to glycopeptides, including T228, T233, and T236, to which Tn antigen is attached in the IgA1 hinge region.

### DETAILED DESCRIPTION OF THE INVENTION

While preferred embodiments of the invention are shown and described herein, such embodiments are provided by way of example only and are not intended to limit the scope of the invention otherwise. Various alternatives to the described embodiments of the invention may be employed in practicing the invention.

Unless otherwise defined, all technical terms used herein have the same meaning as commonly understood by one of ordinary skill in the art to which this invention belongs.

The term “O-linked sugar chain” means a structure in which a sugar chain is bound via an —OH group contained in each amino acid side chain of an amino acid residue of serine (Ser) or threonine (Thr) of a protein. Specific examples of the O-linked sugar chain include T antigen (TF antigen), sialyl T antigen, Tn antigen, sialyl-Tn antigen, and the like.

Examples of the amino acid residue of a polypeptide to which an O-linked sugar chain is bound include an amino acid residue of serine (Ser) or threonine (Thr) in an amino acid sequence of the hinge region of the IgA1 heavy chain polypeptide.

In the present invention, as the amino acid residue of the hinge region polypeptide to which the O-linked sugar chain on the IgA1 heavy chain polypeptide is bound, any of Ser or Thr residues in the amino acid sequence of the hinge region of IgA1 heavy chain polypeptide is available. Examples of these preferably include a sugar chain binding site comprising at least one amino acid residue selected from the group consisting of threonine at position 225, threonine at position 228, serine at position 230, serine at position 232 and threonine at position 236, in the amino acid sequence of human IgA1 heavy chain polypeptide.

The monoclonal antibody of the present invention has a binding activity to the sugar chain-deficient IgA1. The monoclonal antibody of the present invention includes, not limited, an antibody produced by a hybridoma. The anti-sugar chain-deficient IgA1 antibody can be obtained by culturing the hybridoma or administering the hybridoma cell into an animal to cause ascites tumor in the animal and separating and purifying the culture or the ascites.

The animal immunized with an antigen may be any animal, so long as a hybridoma can be prepared, and mouse, rat, hamster, rabbit or the like is suitably used.

The monoclonal antibody is an antibody secreted by a single clone of antibody-producing cells, and recognizes only one epitope (also called antigen determinant) and has the uniformity in amino acid sequence (primary structure).

Examples of the epitope include a single amino acid sequence, a three-dimensional structure consisting of an amino acid sequence, an amino acid sequence having a sugar chain bound thereto, a three-dimensional structure consisting of an amino acid sequence having a sugar chain bound thereto, and the like, which a monoclonal antibody recognizes and binds to. Examples of the epitope of the monoclonal antibody of the present invention include a three-dimensional structure of the sugar chain-deficient IgA1 protein.

Examples of the monoclonal antibody of the present invention include any monoclonal antibody, so long as it recognizes and also binds to the heavy chain hinge region of the sugar chain-deficient IgA1. Specific examples of the monoclonal antibody include monoclonal antibodies 5-2E4E4, 8-E8, 9-E2, 10-D10, 1H5E2, 1H5E7, 1H5F11, and the like.

More specifically, examples of the monoclonal antibody of the present invention include a monoclonal antibody 5-2E4E4 produced by hybridoma 5-2E4E4, a monoclonal antibody which competes with the monoclonal antibody 5-2E4E4 in the binding to the heavy chain hinge region of the sugar chain-deficient IgA1, and a monoclonal antibody that binds to an epitope present in the hinge region of the sugar chain-deficient IgA1 heavy chain to which the monoclonal antibody 5-2E4E4 binds.

Examples of the monoclonal antibody which competes with the monoclonal antibody of the present invention include, specifically, a monoclonal antibody which has a competitive reaction for a variety of monoclonal antibodies and the epitope present in the heavy chain hinge region of the sugar chain-deficient IgA1, as described above.

Optionally, in an exemplary embodiment of the present invention, the monoclonal antibodies of the present invention includes, but is not limited to, NDMC-ASK1 (SEQ ID NO:1), NDMC-ASK2 (SEQ ID NO:2), and NDMC-ASK3(SEQ ID NO:3).

Sequences that can be employed in accordance with the invention are shown herein below:

SEQ ID NO: 1 (NDMC-ASK1):
EVQLQQSGPELVKPGASMKISCKASGYSFTGYTMNWVKQSRGKNLEWIG
LINPYNGVTSYNQKFKGKATLTVDKSSITAYMEFLSLTSEDSAVYYCAR
SGYGNYALAYWGQGTLVTVSA
SEQ ID NO: 2 (NDMC-ASK2):
QIQLVQSGPELKKPGETVKISCKASGYTFTNCGMNWVRQAPGKGLKWMG
WINTYTGKPTYADDFKGRFAFSLETSASTAYLQINNLKNEDMATYFCTK
YGYDPFDYWGQGTTLTVSS
SEQ ID NO: 3 (NDMC-ASK3):
QVQLQQSGPELVKPGASVKISCKASGYTFTSYYIHWVKQRPGQGLEWIG
YIYPRDGSTNYNEKFKGKATLTADTSSSTAYMQLSSLTSEDSAVYFCAR
QLGLPAWFAYWGQGTLVTVSA

A subject may be a human being or a non-human animal, such as cat, dog, rabbit, cattle, horse, sheep, goat, monkey, mouse, rat, gerbil, guinea pig, pig, but is preferably a human.

Optionally, determining binding level to the galactose-deficient IgA1 comprises performing an assay from the group consisting of a Western blot, an enzyme-linked immunosorbent assay (ELISA), an immunoaffinity assay, and a dot-blot assay.

### EXAMPLES

The embodiments encompassed herein are now described with reference to the following examples. These examples are provided for the purpose of illustration only, and the disclosure encompassed herein should in no way be construed as being limited to these examples, but rather should be construed to encompass any and all variations which become evident as a result of the teachings provided herein.

Experimental data were analyzed with GraphPad Prism 7 (GraphPad Software, Inc., CA, USA) or SPSS version 22.0 (IBM, NY, USA). All data are presented as mean ±standard error of mean (SEM). Student's t test (t test) was used in the testing of hypothesis for comparison of means between the groups. A p-value less than 0.05 (typically≤0.05) is statistically significant. Further, ROC curve and logistic regression analyses were plotted by SPSS.

### Example 1

### Aberrantly glycosylated-specific Monoclonal antibody NDMC-ASK2 discriminates normally and abnormally glycosylated IgA1

Hybridoma system was applied to obtain monoclonal antibodies (Mouse anti-human-Gd-IgA1 IgG) that can specifically bind to galactose-deficient glycoform on hinge region of IgA1. To ensure the monoclonal antibody bear the identity to recognize Gd-IgA1 in human serum, the immunogen were customized by purifying IgA1 from human serum, and treated it with enzyme to secure F(ab')2-Gd-IgA1. The immunization, then, used the tailor-made immunogen on BALB/c mice for months and derived arrays of hybridoma clones.

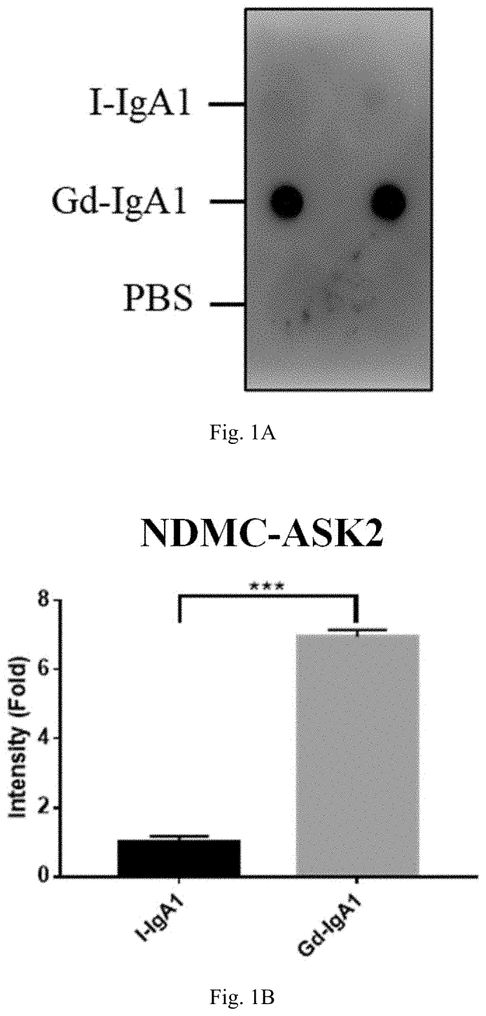

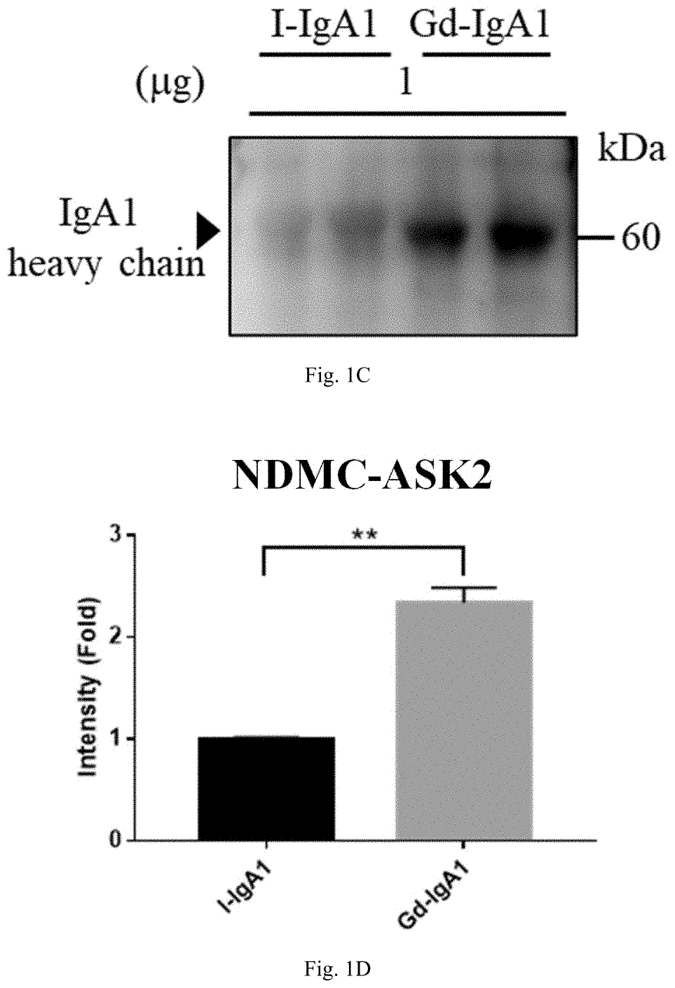

In the process of selecting anti-Gd-IgA1 monoclonal antibody (NDMC-ASK2), we first used enzymatically-generated Gd-IgA1 from plasma-derived IgA1 to examine the supernatant from hybridoma cells in dot blot (FIG. 1A and FIG. 1B) and western blot analysis. The results indicated that NDMC-ASK2 was able to perfectly discriminate I-IgA1 and Gd-IgA1 that were immobilized on NC membrane. The semi-quantitative analysis also showed the significant difference between dividing I-IgA1 and Gd-IgA1 (FIG. 1A and FIG. 1B). The similar results could be observed in western blotting with reducing dye sample treatment. NDMC-ASK2 can specifically recognize galactose-deficient 0-glycan on IgA1 heavy chain rather than intact 0-glycan (FIG. 1C and FIG. 1D).

### Example 2

### Clinical IgAN subjects were unveiled among different type of subjects by NDMC-ASK2 ELISA

In the wake of preliminary test from dot blot and western blot analysis, NDMC-ASK2 was put in clinical binding assay, sandwich ELISA coated with NDMC-ASK2. In plates, serum Gd-IgA1 were captured by immobilized NDMC-ASK2 and subsequently detected by Biotin goat F(ab')2-IgG anti-human IgA and Streptavidin-HRP. Following this assay, the standard curve, which were established by applying a human Gd-IgA1, is acquired.

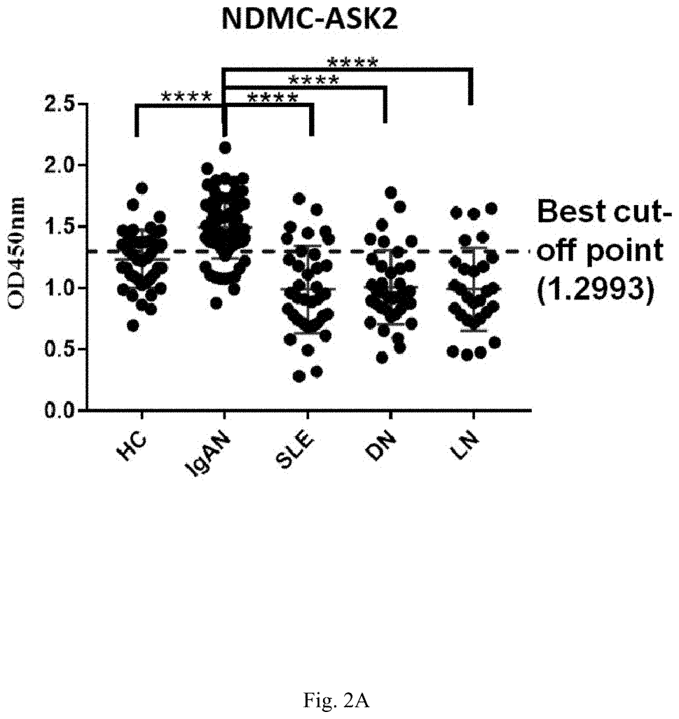

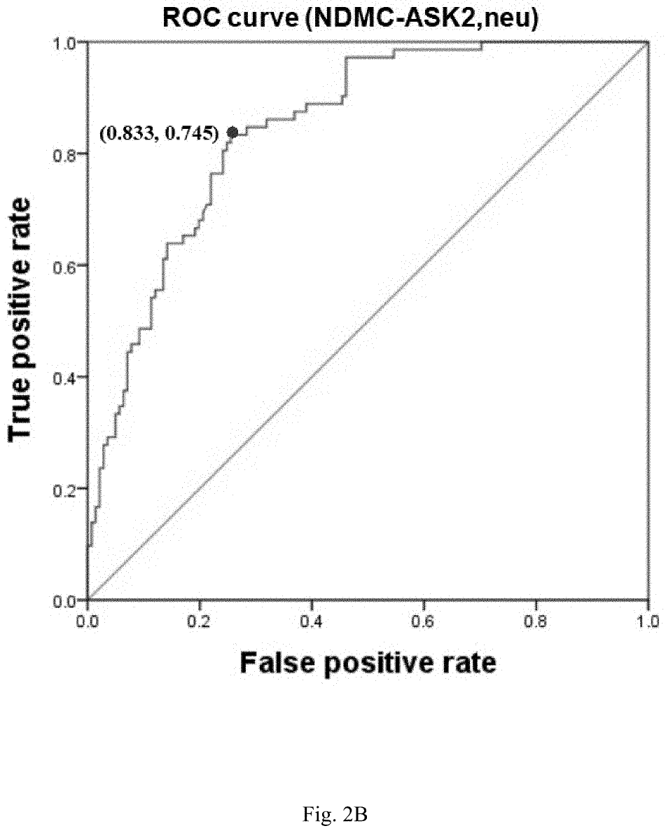

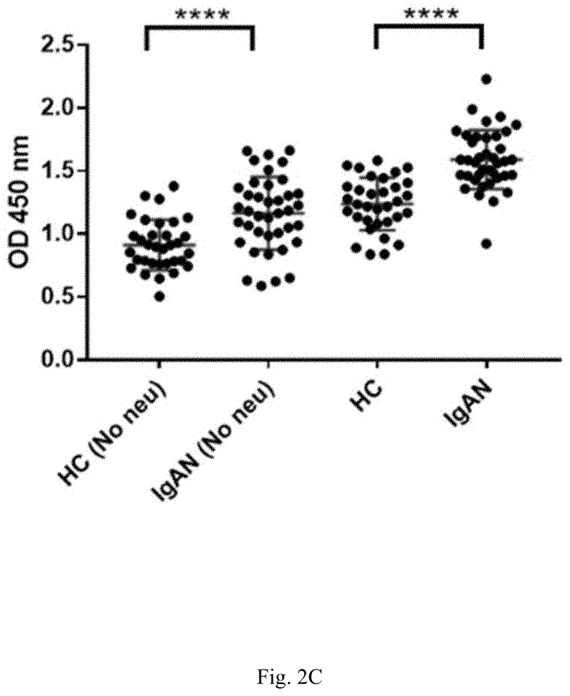

This clinical trial incorporated five different groups, including healthy control (HC), IgAN group, Systemic Lupus Erythematosus group, Lupus Nephritis group, and Diabetic Nephropathy, to be determined NDMC-ASK2′s characteristic of discriminating IgAN patients from healthy group and kidney- and immune-related diseases. the OD value of serum samples were extrapolated from the standard curve and expressed in units/ml. In this embodiment, 1 unit was defined as 1 μg of standard. The result showed that NDMC-ASK2 had superior capability to discriminate IgAN serum samples from others, healthy and disease controls (FIG. 2A-2B). To quantify the distinction of NDMC-ASK2 regarding dividing IgAN and non-IgAN groups, ROC curve was further exploited to derive Area Under the Curve (AUC) and Sensitivity and Specificity. The AUC was up to 0.846, superior capability to identify patients, the sensitivity and specificity were 0.833 and 0.745 based on the best cut-off point on ROC curve (FIG. 2B).

As an illustration, please refer to Table 1, IgAN patients and non-IgAN patients were divided into two groups according to the optimal cut-off value of serum Gd-IgA1, which was obtained by NDMC-ASK2-base ELISA: non-IgAN patients group (Gd-IgA1<1.2993 n=105, 74.5%) and IgAN patients Gd-IgA1 group (Gd-IgA1≥1.2993 μg/mL, n=60, 83.3%).

TABLE 1

| | | | Subject | | |
|---|---|---|---|---|---|
| | | | Non- IgAN | IgAN | Total |
| NDMC-ASK2 | < cut-off value | Count | 105 | 12 | 117 |
| | | % within subjects | 74.5% | 16.7% | 54.9% |
| | >= cut-off value | Count | 36 | 60 | 96 |
| | | % within subjects | 25.5% | 83.3% | 45.1% |
| Total | | Count | 141 | 72 | 213 |
| | | % within subjects | 100.0% | 100% | 100.0% |

According to the sensitivity and specificity this embodiment was acquired, 105 non-IgAN subjects are determined to be negative, with 74.5% True Negative Rate, and 60 IgAN subjects were determined to be positive, with 83.3 True Positive Rate (Table 1).

### EXAMPLE 3

### Comparison of NDMC-ASK2 ELISA and KM55 ELISA for detection of serum Gd-IgA1 levels in subjects with IgAN and non-renal diseases

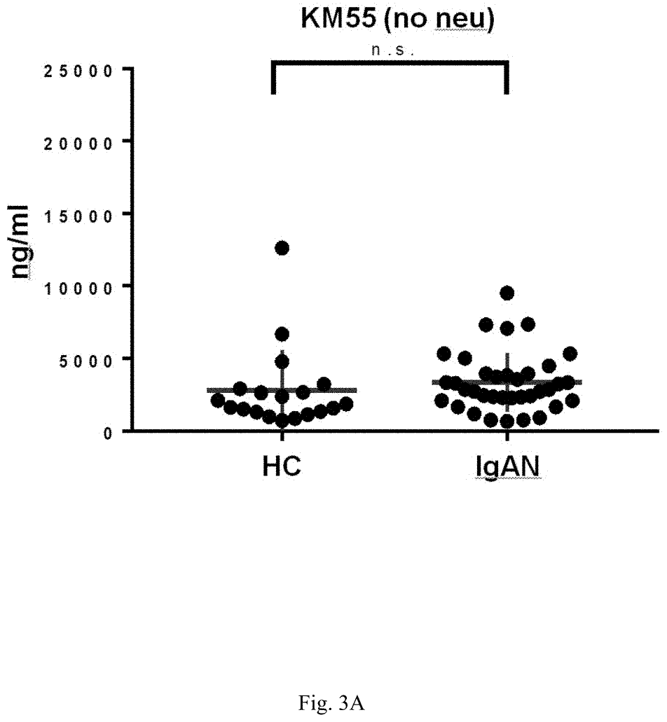

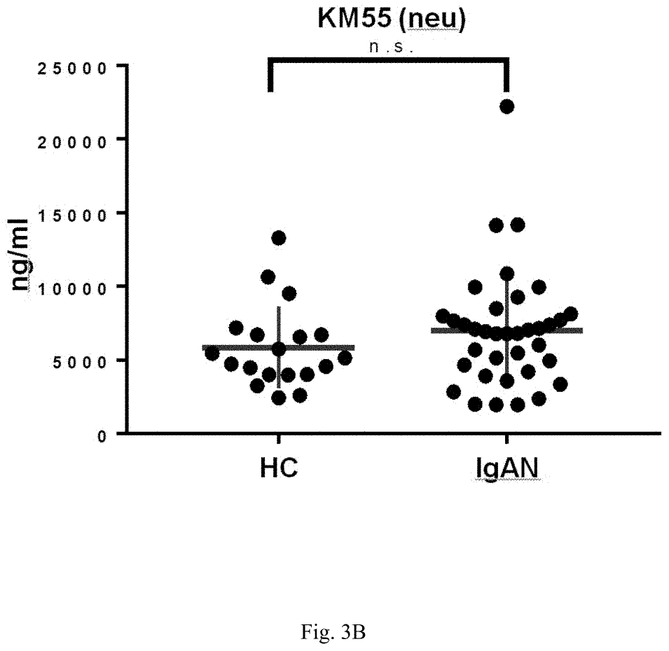

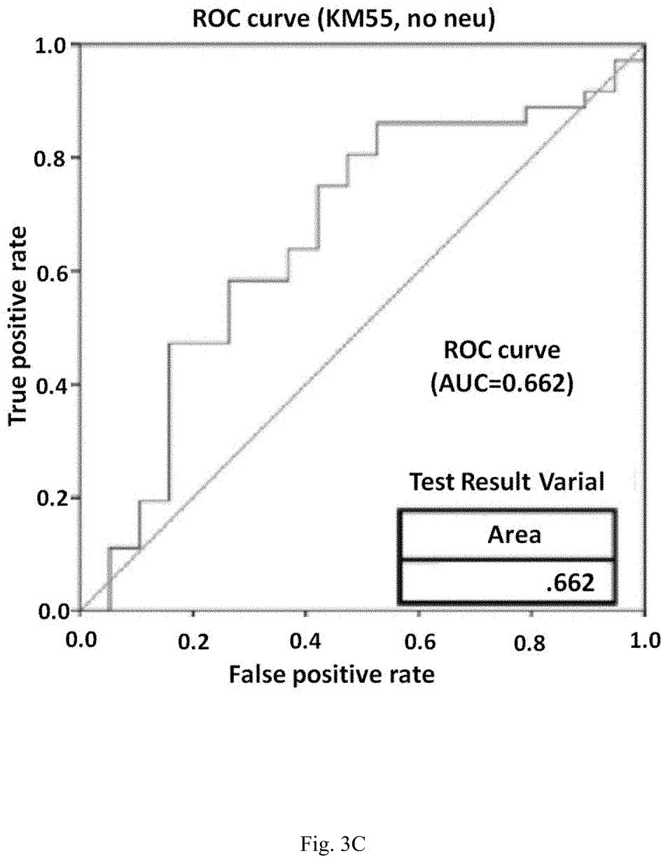

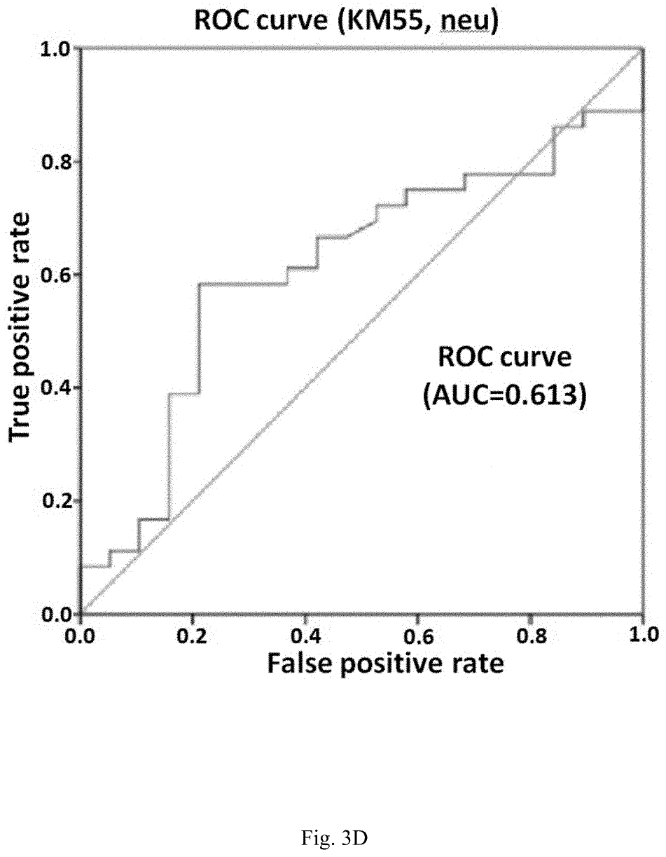

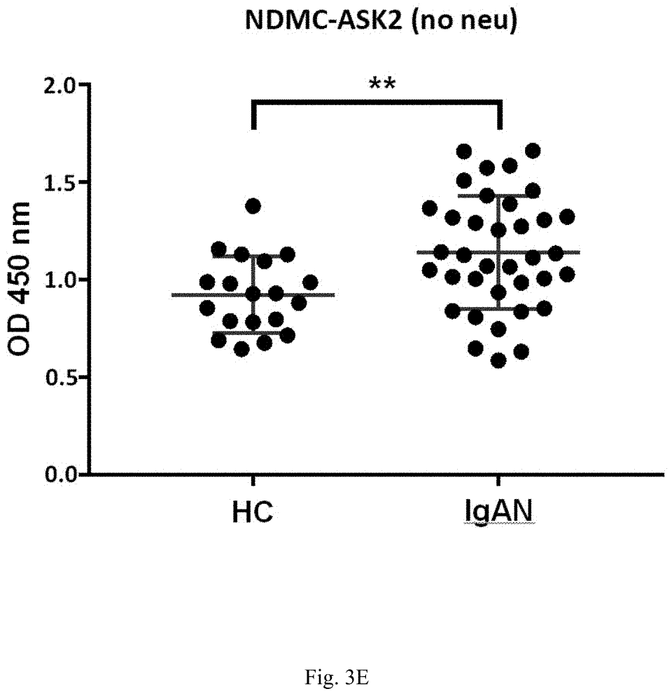

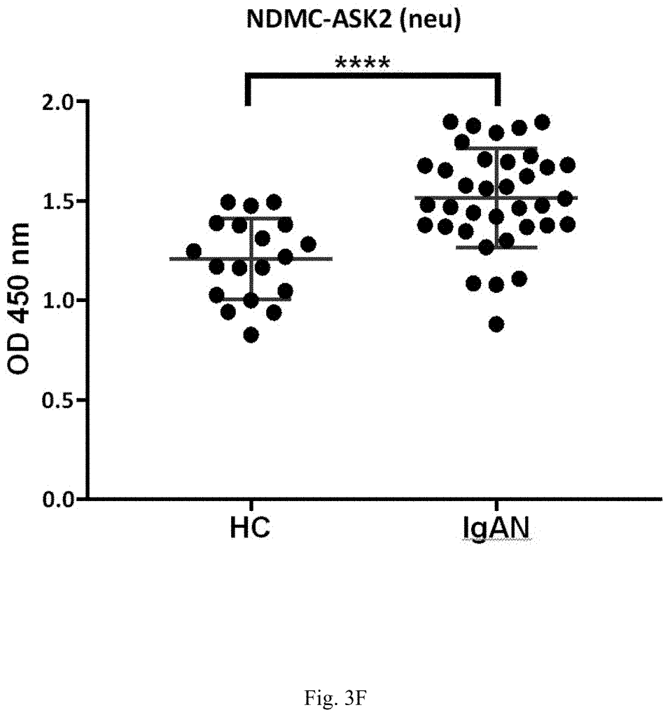

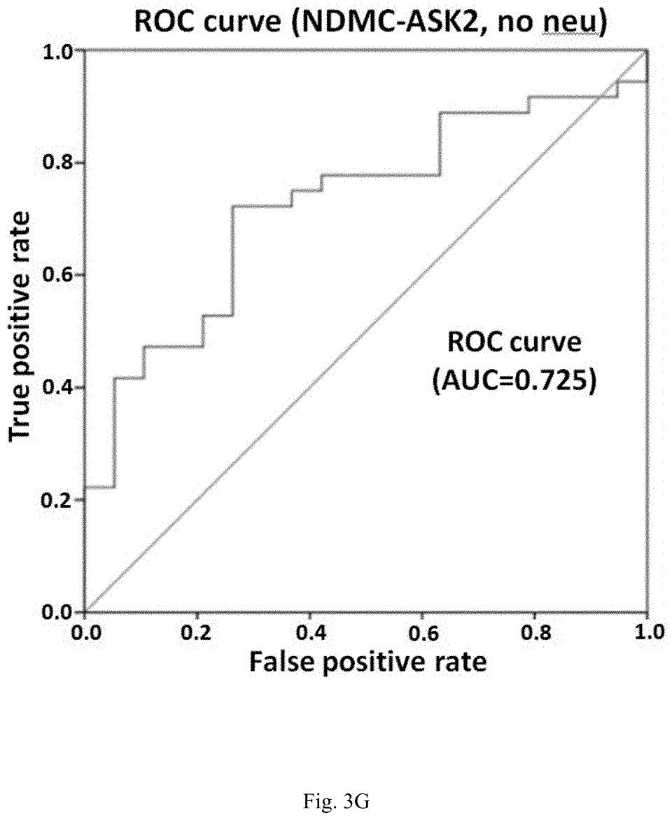

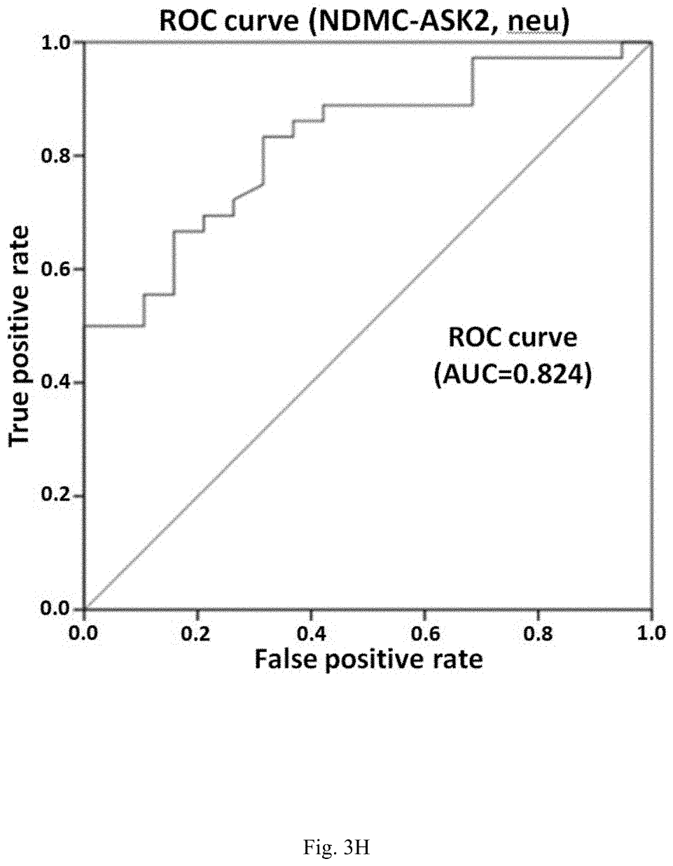

NDMC-ASK2 was further compared with commercially available KM55 (Rat anti-Gd-IgA1 IgG) for serum Gd-IgA1 levels in patients with IgAN and non-renal diseases via measured by ELISA combined with ROC curve analyses. The above-mentioned serum of individuals was treated with or not treated with neuraminidase for ELISA test. The results showed in FIG. 3A-3D that the AUC of KM55 ELISA test was 0.662 by ROC curve analysis under the serum was not treated with neuraminidase (FIG. 3C), and the AUC decreased to 0.613 after the serum was treated with neuraminidase (FIG. 3D). There were no statistically significant differences between HC and IgAN under the serum not treated with neuraminidase or treated with neuraminidase by T test analysis (FIG. 3A-3D).

On the other hand, please refer to FIG. 3E-3H, the AUC of NDMC-ASK2 ELISA test was 0.725 by ROC curve analysis under the serum was not treated with neuraminidase (FIG. 3G), and the AUC increased to 0.824 after the serum was treated with neuraminidase (FIG. 3H).

There were statistically significant differences (p<0.01) between HC and IgAN under the serum not treated with neuraminidase by T test analysis (FIG. 3E, 3G). Further, there were also statistically significant differences (p<0.0001) between HC and IgAN under the serum treated with neuraminidase by T test analysis (FIG. 3F, 3H).

Therefore, it is proved that NDMC-ASK2 ELISA test is far superior to KM55 ELISA test in Gd-IgA1 in serum either not treated with neuraminidase or treated with neuraminidase.

### Example 4

### Circular immune complex detection by NDMC-ASK2

Abundant of Former studies have proved that Gd-IgA1 IgG IC play a vital role in the onset of IgA nephropathy, therefore, a NDMC-ASK2 binding assay for detecting serum Gd-IgA1 IgG immune complex (IC) was established in this embodiment. In plates, serum Gd-IgA1 IgG IC were captured by immobilized NDMC-ASK2 and subsequently detected by goat anti human IgG Fc-HRP. The standard curve, which were also established by applying human Gd-IgA1 and IgG protein, is in this embodiment.

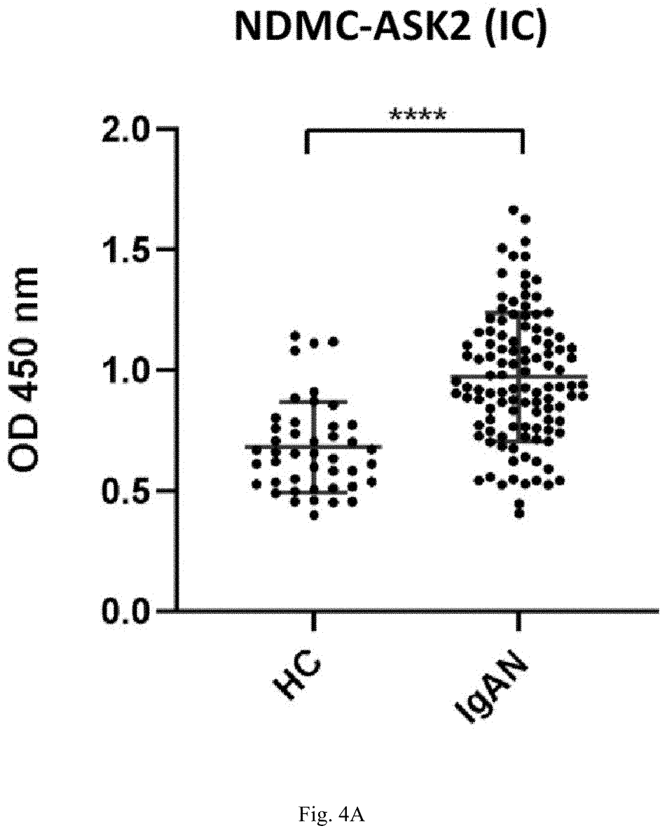

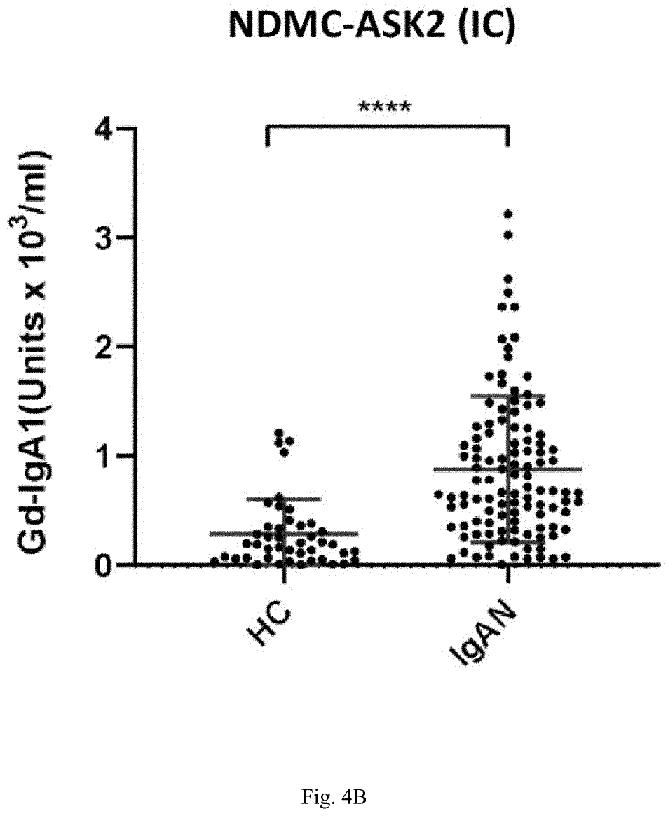

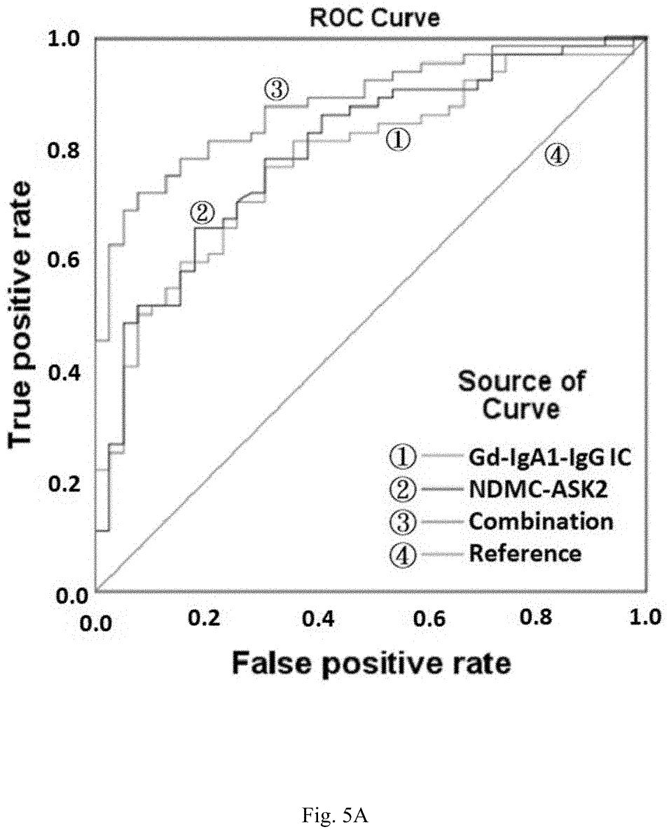

Please refer to FIG. 4A and FIG. 4B, the result of this binding assay indicates NDMC-ASK2 can significantly divide health control (n=43) and IgAN patients (n=112) by detecting serum Gd-IgA1 IgG IC. The OD value of serum samples were extrapolated from the standard curve and expressed in units/ml. In this embodiment, 1 unit was defined as 1 μg of standard. This finding suggests that not only could NDMC-ASK2 bind to serum Gd-IgA1, but also bear the nature to detect IC forms in serum.

### Example 5

### Combined NDMC-ASK2 and IC analysis increased sensitivity and specificity by ROC curve and logistic regression analyses

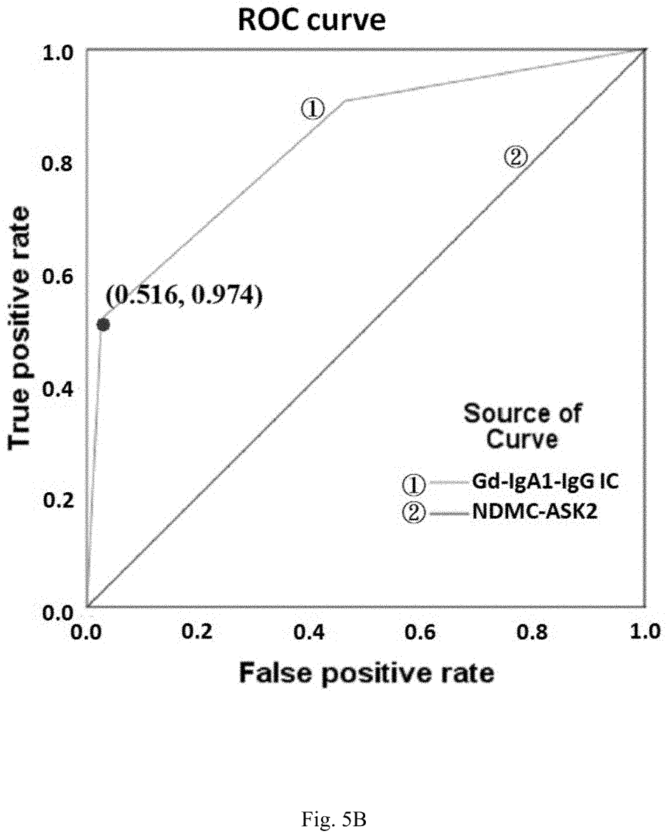

A combined NDMC-ASK2 and partial immune-complex with NDMC-ASK2 assay were established by combining ROC curve and logistic regression analyses, which helps increase the sensitivity to 0.906 although the specificity is 0.974 (FIG. 5A-5B, Table 2 and Table 3). Based on these results, the combined NDMC-ASK2 with Gd-IgA1-IgG IC can be further developed as promising diagnostic reagents for IgA in this invention.

TABLE 2
Area Under the Curve.

| Test Result Variable (s) | Area |
|---|---|
| Gd-IgAa-IgG IC | 0.778 |
| NDMC-ASK2 | 0.797 |
| Combination | 0.880 |

TABLE 3

| Positive if greater | | |
|---|---|---|
| than or equal to | Sensitivity | Specificity |
| One positive | 0.906 | 0.538 |
| Two positive | 0.516 | 0.974 |

### Example 6

### Clinical IgAN patients were unveiled among different type of subjects by NDMC-ASK1 ELISA

In this embodiment, NDMC-ASK1 was put in clinical binding assay, sandwich ELISA coated with NDMC-ASK1. In plates, serum Gd-IgA1 were captured by immobilized NDMC-ASK1 and subsequently detected by Biotin goat F(ab')2-IgG anti-human IgA and Streptavidin-HRP. Following this assay, the standard curve, which were established by applying a human Gd-IgA1, is acquired.

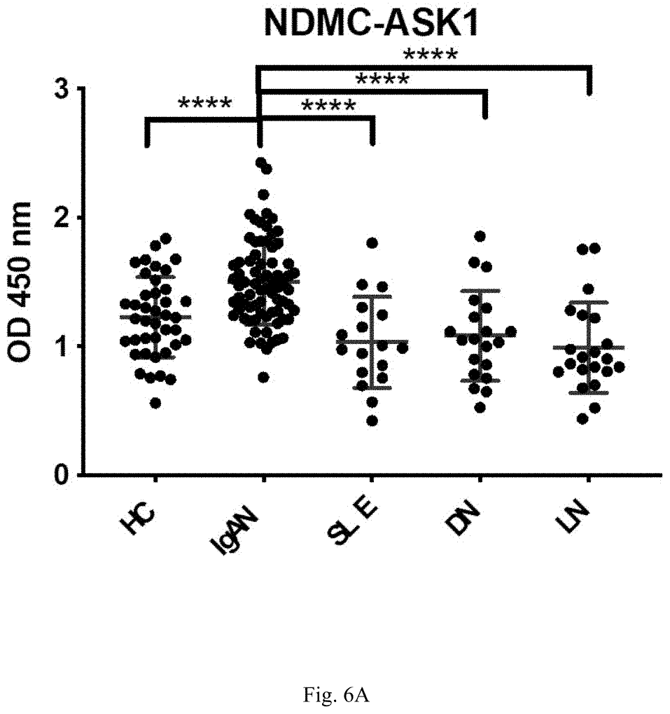

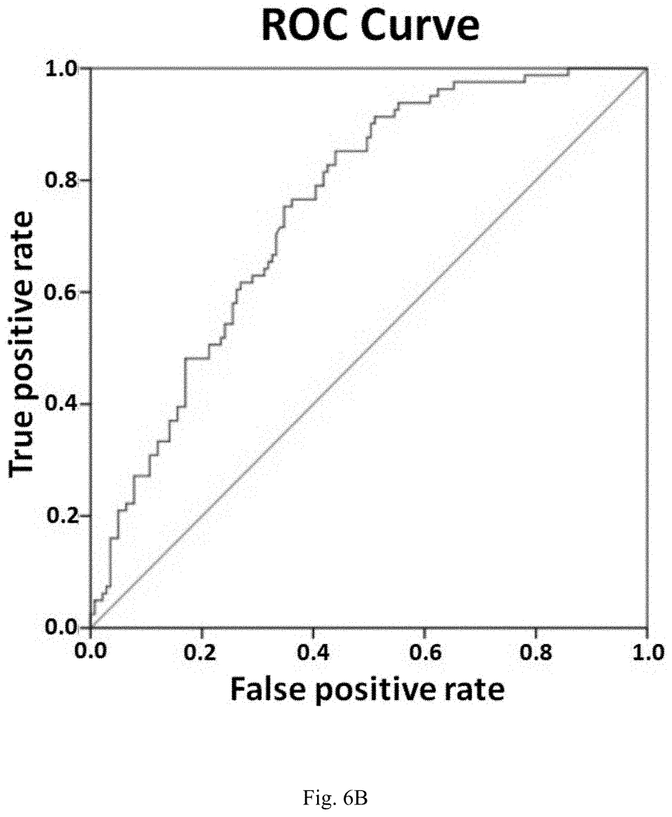

This clinical trial incorporated five different groups, including healthy control (HC), IgAN group, Systemic Lupus Erythematosus group, Lupus Nephritis group, and Diabetic Nephropathy, to determined NDMC-ASK1's characteristic of discriminating IgAN patients from healthy group and kidney- and immune-related diseases. the OD value of serum samples were extrapolated from the standard curve and expressed in units/ml. In this embodiment, 1 unit was defined as 1 μg of standard. The result showed that NDMC-ASK1 had superior capability to discriminate IgAN serum samples from others, healthy and disease controls (FIG. 6A-6B). To quantify the distinction of NDMC-ASK1 regarding dividing IgAN and non-IgAN groups, ROC curve was further exploited to derive Area Under the Curve (AUC) and Sensitivity and Specificity. The AUC was up to 0.751 (FIG. 6B). The sensitivity and specificity were 0.852 and 0.560 based on the best cut-off point on ROC curve (FIG. 6B)

### Example 7

### Clinical IgAN patients were unveiled among different type of subjects by NDMC-ASK3 ELISA

In this embodiment, NDMC-ASK3 was put in clinical binding assay, sandwich ELISA coated with NDMC-ASK3. In plates, serum Gd-IgA1 were captured by immobilized NDMC-ASK3 and subsequently detected by Biotin goat F(ab')2-IgG anti-human IgA and Streptavidin-HRP. Following this assay, the standard curve, which were established by applying a human Gd-IgA1 protein, is acquired.

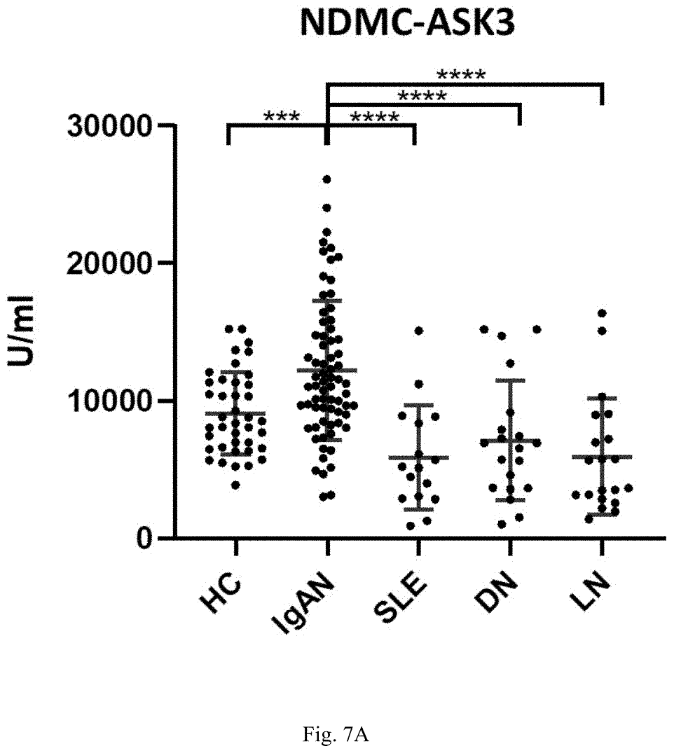

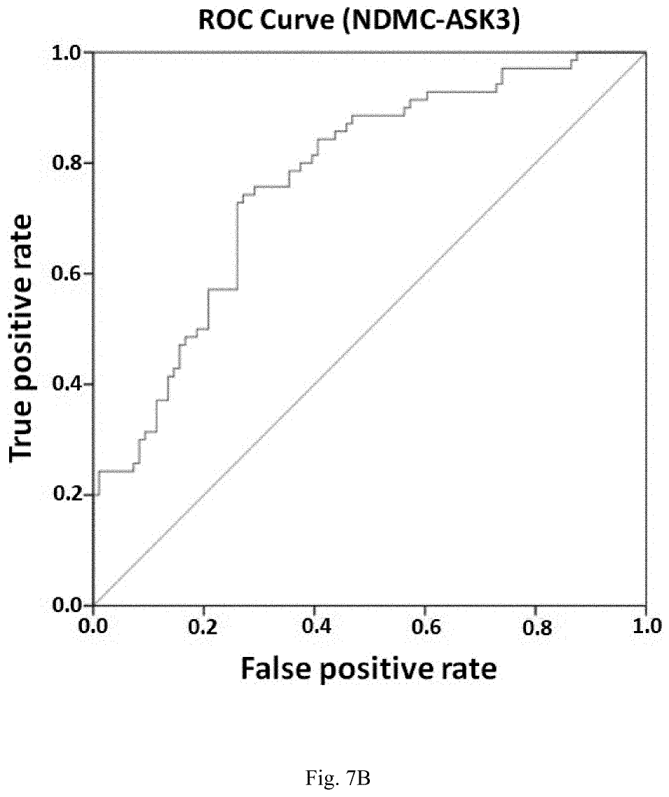

This clinical trial incorporated five different groups, including healthy control (HC), IgAN group, Systemic Lupus Erythematosus group, Lupus Nephritis group, and Diabetic Nephropathy, to determined NDMC-ASK3′s characteristic of discriminating IgAN patients from healthy group and kidney- and immune-related diseases. the OD value of serum samples were extrapolated from the standard curve and expressed in units/ml. In this embodiment, 1 unit was defined as 1μg of standard. The result showed that NDMC-ASK3 had superior capability to discriminate IgAN serum samples from others, healthy and disease controls (FIG. 7A-7B). To quantify the distinction of NDMC-ASK3 regarding dividing IgAN and non-IgAN groups, ROC curve was further exploited to derive Area Under the Curve (AUC) and Sensitivity and Specificity. The AUC was up to 0.77 (FIG. 7B).

As an illustration, please refer to Table 4, IgAN patients and non-IgAN patients were divided into two groups according to the optimal cut-off value of serum Gd-IgA1, which was obtained by NDMC-ASK3-base ELISA: non-IgAN patients group (Gd-IgA1<9165.4625 n=70, 72.9%) and IgAN patients Gd-IgA1 group (Gd-IgA1>9165.4625, n =52, 74.3%).

TABLE 4

| | | | Subject | | |
|---|---|---|---|---|---|
| | | | Non- IgAN | IgAN | Total |
| NDMC-ASK3 | cut-off value < | Count % | 70 | 18 | 88 |
| | 9165.4625 | within subjects | 72.9% | 25.7% | 53.0% |
| | cut-off value >= | Count % | 26 | 52 | 78 |
| | 9165.4625 | within subjects | 27.1% | 74.3% | 47.0% |
| Total | | Count % | 96 | 70 | 166 |
| | | within subjects | 100.0% | 100% | 100.0% |

### EXAMPLE 8

### The epitopes and pathogenic form on hinge region of IgA1 recognized by NDMC-ASK2, NDMC-ASK1 and NDMC-ASK3

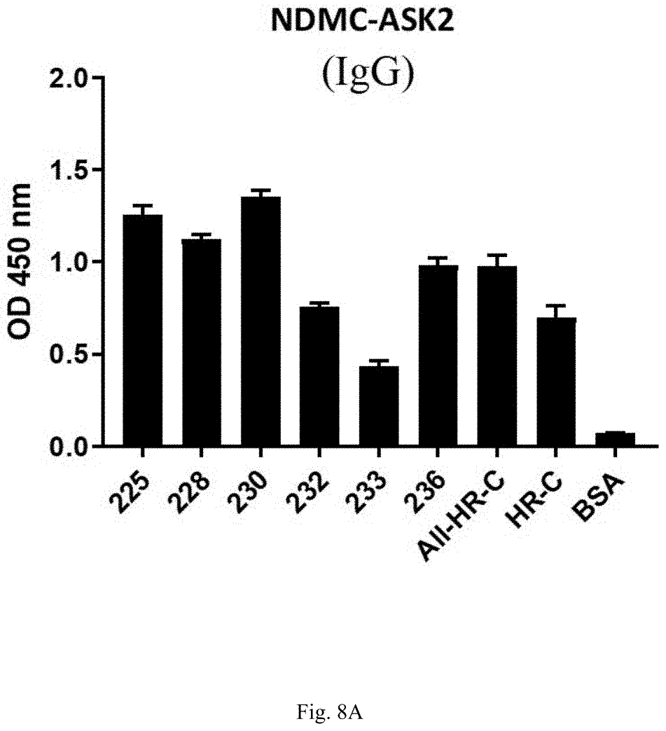

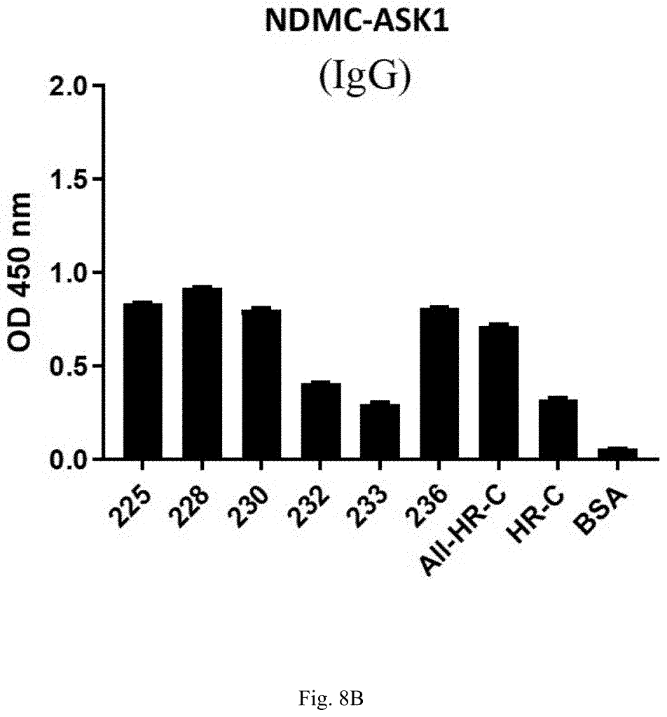

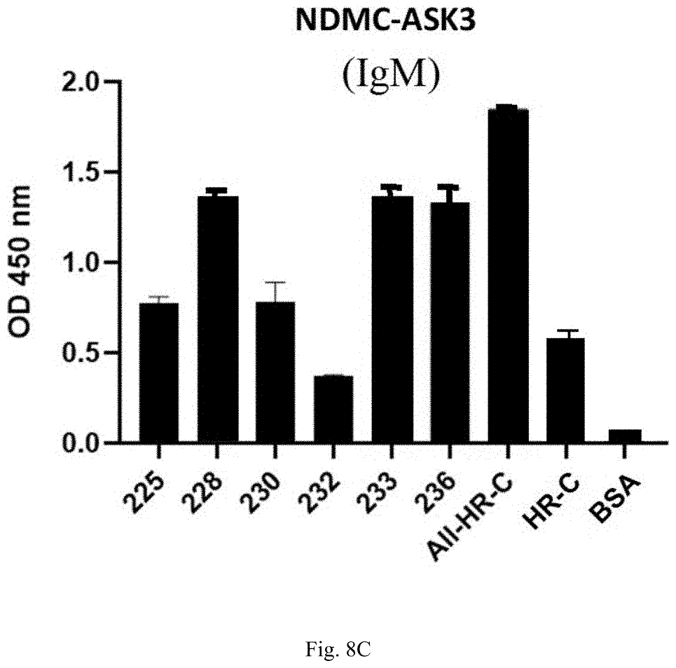

To figure out the detailed recognition characteristic of NDMC-ASK2, a series of glycopeptides, with six different single GalNAc-modified hinge region peptides, previous study reported to observe aberrant glycosylation on human IgA1 in serum, were synthesized and conjugated with BSA, which were employed in binding assay. The result indicated that NDMC-ASK2 strongly bound to Thr225—, Thr228—, Ser230—, Thr236—, and All-GalNAc-attached peptide, whereas only weak recognition on Ser232, Thr233, and pure peptide (FIG. 8A). Therefore, NDMC-ASK2 can specifically bind to Gd-IgA1 with exposed Thr225—, Thr228—, Ser230—, Thr236—, and All-GalNAc motif in this embodiment.

Meanwhile, the result indicated that NDMC-ASK1 would recognize on Ser232, Thr233, and pure peptide (FIG. 8B).

On the other hand, the result indicated that NDMC-ASK3 strongly bound to Thr228—, Thr233—, Thr236—, and All-GalNAc-attached peptide, whereas only weak recognition on Ser232, and pure peptide (FIG. 8C). Therefore, NDMC-ASK3 can specifically bind to Gd-IgA1 with exposed Thr228—, Thr233—, Thr236—, and All-GalNAc motif in this embodiment.

To sum up, this invention disclosed that (1) after several times of immunization on BALB/c mice for months, B cells were took from the spleen to manufacture hybridoma cells and conducted limiting dilution to obtain single hybridoma cell subsequently. Further examinations were conducted to test the binding ability of monoclonal antibodies with Gd-IgA1; (2) NDMC-ASK2 can specifically bind with Gd-IgA1 at dot blot analysis, Western blot analysis, and Enzyme-linked immunosorbent assay (ELISA); (3) NDMC-ASK2 can well discriminate IgAN patients from healthy controls (HCs), without treating sera with neuraminidase for desialylation of glycans. Importantly, when serum samples were treated with neuraminidase, the sensitivity involving the ELISA-based analysis with NDMC-ASK2 was shown to be greatly enhanced, with a AUC value as: 0.884; sensitivity as: 0.872 and specificity as: 0.75; (4) compared with KM55, a published rat IgG anti-Gd-IgA1, which binds to T225 and T233 only, NDMC-ASK2 was able to bind with a much broad range of sites, including T225, T228, S230, S232, T233 and T236 to which Tn antigen is attached on the hinge region of IgA1; (5) NDMC-ASK2 exhibited significantly higher sensitivity and specificity in differentiating HCs from IgAN patients than KM55; (6) Detection of serum IgG autoantibodies using a combined immune-complex (IC)-based assay and NDMC-ASK2-based ELISA analysis was shown to significantly increased the specificity of discriminating IgAN from HCs. Notably, a combined NDMC-ASK2 and IC-based assay detected by NDMC-ASK2-based assay helped increase sensitivity to 0.913 and specificity 0.667; (7) IgM (NDMC-ASK3) can well discriminate IgAN patients from HCs. In other words, the results form combined serum levels of Gd-IgA1 and IC-based assays an example to fustily NDMC-ASK-based assay, especially NDMC-ASK2-based assay can be further developed as promising rapid, early, non-invasive diagnostic kits for IgAN soon. (please refer to Table 5-Table 8; AUC: Area under the curve; n.s.:No signifince; BLI: Bio-layer interferometry; KD: Affinity constant; Kon: association rate constant; Kdis: dissociation rate constant.).

TABLE 5

Neu-treated serum
(HC: n = 19; IgAN: n = 36)

| | Mean (SD) | | |
|---|---|---|---|
| | (OD 450 nm or ng/ml) | | AUC |
| NDMC-ASK2 | 1.208 (0.203) | P < 0.0001 | 0.824 |
| | 1.515 (0.249) | | |
| KM55 | 5837.905 (2793.477) | n.s. | 0.613 |
| | 6992.056 (3973.910) | | |

TABLE 6

| | Intact serum | | |
|---|---|---|---|
| | (HC: n = 19; IgAN: n = 36) | | |
| | Mean (SD) | | |
| | (OD 450 nm or ng/ml) | | AUC |
| NDMC-ASK2 | 0.923 (0.195) | P < 0.001 | 0.725 |
| | 1.139 (0.292) | | |
| KM55 | 2785.312 (2790.280) | n.s. | 0.662 |
| | 3353.471 (2029.171) | | |

TABLE 7

| | BLI | | |
|---|---|---|---|
| | KD (M) | Kon (1/Ms) | Kdis (1/s) |
| NDMC-ASK2 | 1.83 × 10⁻⁶ | 4.6 × 10⁴ | 8.4 × 10⁻² |
| KM55 | 7.4 × 10⁻⁸ | 1.68 × 10⁴ | 1.24 × 10⁻³ |

TABLE 8

| | GalNAc moiety epitope | | | | | | | |
|---|---|---|---|---|---|---|---|---|
| | T225 | T228 | S230 | S232 | T233 | T236 | All-HR-C | Immunogen |
| NDMC-ASK2 | + | + | + | − | − | + | + | Gd-Fab |
| KM55 | + | − | − | − | + | − | + | Glycopeptide |

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed is:

1. A kit for unraveling the mechanisms of IgA nephropathy and establishing therapeutical strategies, comprising an antibody.

2. A kit according to claim 1, wherein the antibody is acquired from using a human Gd-IgA1 protein system.

3. A kit according to claim 2, wherein the 6^{th} to 11^{th} amino acids counted from the N-terminal of the antibody are QSGPEL, wherein the 19^{th} to 27^{th} amino acids counted from the N-terminal of the antibody are KISCKASGY.

4. A kit according to claim 3, wherein the antibody is select from NDMC-ASK1 (SEQ ID NO: 1), NDMC-ASK2 (SEQ ID NO: 2), and NDMC-ASK3 (SEQ ID NO: 3).

5. A method for detecting galactose-deficient IgA1 in a subject, comprising:
(a) obtaining a biological sample from a subject; and
(b) using an antibody in an immunoassay to detect a galactose-deficient glycopeptide of IgA1 in the subject.

6. A method according to claim 5, wherein the 6^{th} to 11^{th} amino acids counted from the N-terminal of the antibody are QSGPEL, wherein the 19^{th} to 27^{th} amino acids counted from the N-terminal of the antibody are KISCKASGY.

7. A method according to claim 6, wherein the antibody is select from NDMC-ASK1 (SEQ ID NO: 1), NDMC-ASK2 (SEQ ID NO: 2), and NDMC-ASK3 (SEQ ID NO: 3).

8. A method according to claim 7, wherein the biological sample is selected from plasma, serum, or blood; wherein the subject is selected from IgA nephropathy (IgAN) subjects, lupus nephritis (LN) subjects, and healthy subjects.

9. A method of diagnosing IgA nephropathy in a subject, comprising:
(a) using an antibody in an immunoassay; and
(b) determining whether the antibody binds to a galactose-deficient glycopeptides of IgA1, binding of the antibody to the galactose-deficient glycopeptide of IgA1 indicating the subject has or is at risk of developing IgA nephropathy.

10. A method according to claim 9, wherein the 6^{th} to 11^{th} amino acids counted from the N-terminal of the antibody are QSGPEL, wherein the 19^{th} to 27^{th} amino acids counted from the N-terminal of the antibody are KISCKASGY.

11. A method according to claim 10, wherein the antibody is select from NDMC-ASK1 (SEQ ID NO: 1), NDMC-ASK2 (SEQ ID NO: 2), and NDMC-ASK3 (SEQ ID NO: 3).

12. A method according to claim 11, wherein the biological sample is selected from plasma, serum, or blood.

13. A method for monitoring a subject undergoing a treatment or therapy for an immune response to determine whether the subject is responsive to the treatment or therapy comprising detecting a level of expression, activity and/or function of a biomarker or a biomarker in the absence of the treatment or therapy and comparing the level of expression, activity and/or function of a biomarker in the presence of the treatment or therapy, wherein a difference in the level of expression, activity and/or function of the biomarker in the presence of the treatment or therapy indicates whether the patient is responsive to the treatment or therapy, wherein the biomaker is a galactose-deficient glycopeptide on hinge region of IgA1 and wherein the biomaker is specific for an antibody.

14. A method according to claim 13, wherein the 6^{th} to 11^{th} amino acids counted from the N-terminal of the antibody are QSGPEL, wherein the 19^{th} to 27^{th} amino acids counted from the N-terminal of the antibody are KISCKASGY.

15. A method according to claim 14, wherein the galactose-deficient glycopeptide is select from Thr225, Thr228, Ser230, Ser232, Thr233, Thr236, or All-GalNAc motif.
